SEQUENCE LISTING (4 sequences)

SEQ ID NO: 1 - DNA, 1377 bp, Pyrococcus horikoshii
atggaggggaatactattcttaaaatcgtactaatttgcactattttagcaggcctattcgggcaagtcgtgccagtatatgcagaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttcaagtactcaatccgttattcggagtaccacccctacaaagtcaaatacaagtaagaagatttgtggaccagcaattcttatcatcctagcagtattctctcttctcttaagaagggctcccaggtag

SEQ ID NO: 2 - protein, 458 aa, Pyrococcus horikoshii
MEGNTILKIVLICTILAGLFGQVVPVYAENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSSSSTQSVIRSTTPTKSNTSKKICGPAILIILAVFSLLLRRAPR

SEQ ID NO: 3 - DNA, 43 bp, Artificial Sequence
  Description of Artificial Sequence PCR primer
ttttgaattctttcatatggaggggaatactattcttaaaatc

SEQ ID NO: 4 - DNA, 46 bp, Artificial Sequence
  Description of Artificial Sequence PCR primer
tttttctagatttggatcctttgggctacctgggagcccttcttaa

(19) United States
(12) Patent Application Publication
(10) Pub. No.: US 2003/0129723 A1
(43) Pub. Date: Jul. 10, 2003

(54) Thermophilic endoglucanase

(75) Inventors: Kazuhiko Ishikawa, Osaka (JP); Hiroyasu Ishida, Ibaraki (JP); Yoshitsugu Kosugi, Ibaraki (JP); Susumu Ando, Osaka (JP); Akio Shimomura, Shiga (JP)

Correspondence Address: ARMSTRONG,WESTERMAN & HATTORI, LLP, 1725 K STREET, NW, SUITE 1000, WASHINGTON, DC, 20006, US

(73) Assignee: RAKUTO KASEI INDUSTRIAL CO., LTD., Otsu-shi (JP)

(21) Appl. No.: 10/293,344

(22) Filed: Nov. 14, 2002

(30) Foreign Application Priority Data
Nov. 14, 2001 (JP) 2001-349328
Oct. 9, 2002 (JP) 2002-295578

(51) Int. Cl. C12N 9/24; C07H 21/04; C12P 21/02; C12N 1/21; C12N 15/74; C12N 5/06; C12N 5/04

(52) U.S. Cl. 435/200; 435/69.1; 435/252.3; 435/320.1; 536/23.2; 435/325; 435/419; 435/348

(57) ABSTRACT

The present invention provides a polypeptide that has a thermophilic endoglucanase activity, a DNA that comprises a nucleotide sequence encoding the polypeptide, a recombinant DNA and an expression vector that comprise the DNA, a transformant that is transformed by the expression vector, and a method of producing the polypeptide using the transformant. The endoglucanase of the present invention shows a maximum activity at a high temperature range around 97° C., so that it is industrially useful.

### BACKGROUND OF THE INVENTION

1. Field of the Invention

The present invention relates to a novel thermophilic endoglucanase, a DNA encoding the thermophilic endoglucanase, a transformant that is transformed by the DNA encoding the thermophilic endoglucanase, and a method of producing a thermophilic endoglucanase using the transformant.

2. Description of Related Art

Cellulose is homopolysaccharide in which D-glucose units are linked by β-1,4 bonds in the form of a straight chain and is present extensively in the natural world in the form of crystal or non-crystal. It is bonded together intricately with lignin, hemicelluloses, and pectins to form plant tissues.

Cellulase is a general term for enzymes that catalyzes enzyme reaction system that hydrolyzes cellulose to cellooligosaccharide, then to cellobiose, and finally to glucose. Cellulase is produced by fungi, actinomyces, myxobacteria, various bacteria containing eubacteria, or a plant. Particularly, it is known that the cellulose produced by filamentous acremonium cellulolyticus has a strong saccharifying ability. Therefore, cellulase is useful as livestock food and silage, as is disclosed in Japanese Unexamined Patent Publication Nos. (Patent Kokai Nos.) 04-117244 (1992) and 07-236431 (1995). Thus, cellulase having a wide variety of specificity has been identified so far.

Cellulase are very important for industrial use. For example, it can be used as a component contained in a detergent composition or a fabric softener. Also, it can be used for treating cellulose fiber or cellulose fabric, for biopolishing new fabric (enzymatic finishing), and for stonewashing cellulose-containing fabric, especially denim, to improve the appearance.

Further, cellulase can be used for cleaning of waste water or deinking of used paper in paper pulp treatment.

Cellulase is classified broadly into three families: endoglucanase, exoglucanase, and β-glucosidase. Among them, endoglucanase (endo β-1,4-glucanase (EC 3.2.1.4)) is a effective enzyme for hydrolyzing β-1,4-glucoside bonds between D-glucosides that are constituents of cellulose under physiological conditions. Endoglucanase catalyzes the endohydrolysis of cellulose, cellulose derivatives (e.g. carboxymethylcellulose and hydroxyethylcellulose), 1,4-β-D-glucoside bond in lichenin, or β-1,4 bond in other plant material containing mixed β-1,3-glucane such as β-D-glucane, xyloglucane, and cellulose of crops.

However, almost all the known endoglucanases have an optimum temperature of around 35° C. to 60° C. At present, a mold-derived enzyme having a low heat resistance has been used for treating crystalloid cellulose and cellulose fibers. From the viewpoint of reaction efficiency, it is preferable to conduct a reaction at much higher temperature. For this reason, endonuclease having an optimum temperature of 75° C. or more has been developed (which is disclosed in Japanese Unexamined Patent Publication No. (Patent Kokai No.) 11-75849 (1999)). However, in order to remove impure materials and to prevent effects of impure materials on reaction when an enzyme is obtained by genetic engineering techniques and when cellulose is treated using the thus-obtained enzyme, it is greatly desired to develop endoglucanase that is stable and holds a catalytic activity at a high temperature close to 100° C. Such endoglucanase is hereinafter referred to as “thermophilic endoglucanase”.

### SUMMARY OF THE INVENTION

As a result of our researches to overcome the above disadvantages, we have focused attention on hyperthermophilic archaeon that grows at 90 to 100° C., and we have eventually found a novel thermophilic endoglucanase from thermophile Pyrococcus horikoshii (JCM9974) that grow at a high temperature (100° C. or more).

Accordingly, an object of the present invention is to provide a novel thermophilic endoglucanase, an amid acid sequence thereof, and a DNA comprising a nucleotide sequence encoding the thermophilic endoglucanase. Further, another object of the present invention is to provide an expression vector containing the DNA, transformant containing the vector, and a method of producing such thermophilic endoglucanase.

According to the present invention, there is provided a novel endoglucanase having the following properties:

(a) a molecular weight of about 43 kilo daltons;

(b) an optimum temperature of 95° C. or more and 100° C. or less:

(c) an optimum pH of 5.4 to 6.0; and

(d) stability at about 97° C. for 3 hours.

According to the present invention, a thermophilic endoglucanase comprises an amino acid sequence as shown in positions 1-458 of SEQ ID NO: 2.

According to the present invention, a thermophilic endoglucanase has a thermophilic endoglucanase activity and comprises an amino acid sequence as shown in positions 1-458 of SEQ ID. NO: 2 in which 1 to 100 amino acid residue(s) is/are missing, substituted, or added.

According to the present invention, a thermophilic endoglucanase comprises an amino acid sequence as shown in positions 29-458 of SEQ ID NO: 2.

According to the present invention, a thermophilic endoglucanase has a thermophilic endoglucanase activity and comprises an amino acid sequence as shown in positions 29-458 of SEQ ID NO: 2 in which 1 to 100 amino acid residue(s) is/are missing, substituted, or added.

In one embodiment, the thermophilic endoglucanase is derived from prokaryote.

In another embodiment, the thermophilic endoglucanase is derived from Pyrococcus.

In still another embodiment, the thermophilic endoglucanase is derived from Pyrococcus horikoshii.

According to the present invention, a DNA comprises a polynucleotide sequence encoding a thermophilic endoglucanase comprising an amino acid sequence as shown in positions of 1-458 of SEQ ID NO: 2.

According to the present invention, a DNA comprises a polynucleotide sequence encoding a thermophilic endoglucanase that has a thermophilic endoglucanase activity and that comprises an amino acid sequence as shown in positions of 1-458 of SEQ ID NO: 2 in which 1 to 100 amino acid residue(s) is/are missing, substituted, or added.

According to the present invention, a DNA comprises a polynucleotide sequence encoding a thermophilic endoglucanase comprising an amino acid sequence as shown in positions of 29 to 458 of SEQ ID NO: 2.

According to the present invention, a DNA comprises a polynucleotide sequence encoding a thermophilic endoglucanase that has a thermophilic endoglucanase activity and that comprises an amino acid sequence as shown in position of 29-458 of SEQ ID NO: 2 in which 1 to 100 amino acid residue(s) is/are missing, substituted, or added.

According to the present invention, a DNA comprises a polynucleotide sequence encoding a thermophilic endoglucanase derived from Pyrococcus horikoshii.

According to the present invention, a DNA comprises a polynucleotide sequence as shown in positions of SEQ ID NO: 1 from nucleotide 1 to nucleotide 1374.

According to the present invention, a DNA comprises a polynucleotide sequence as shown in SEQ ID NO: 1 from nucleotide 85 to nucleotide 1374.

According to the present invention, a DNA encodes a polypeptide that can be hybridized with a polynucleotide sequence as shown in SEQ ID NO:1 from nucleotide 85 to nucleotide 1377 under stringent conditions and that has a thermophilic endoglucanase activity.

According to the present invention, a recombinant vector comprises any one of the aforementioned DNA.

According to the present invention, an expression vector comprises any one of the aforementioned DNAs in a site to be cntrolled by a promoter for expressing a thermophilic endoglucanase and has an ability of expressing a polypeptide that has a thermophilic endoglucanase activity in a host cell.

According to the present invention, a transformant is transformed by any one of the aforementioned vector.

In one embodiment, a host cell of the aforementioned transformants is a bacteria cell, animal cell, plant cell, or insect cell.

According to the present invention, a method of producing a polypeptide having a thermophilic endoglucanase activity comprises the steps of: incubating any one of the aforementioned transformant; and extracting a thermophilic endoglucanase from a culture.

The present invention can provide a novel thermophilic endoglucanase that has an optimum reaction temperature of 97° C. or more and that catalyzes the selective hydrolytic splitting of cellulose or cellulose derivatives. Further, since enzyme molecules are stable, an organic solvent resistance can be expected to be increased. The enzyme of the present invention can be used as a surfactant for transforming a plant biomass into fuel or chemical substances, as a treatment agent for cellulose or fibers containing cellulose, or as a treatment agent for clarifying and extracting beverages such as juice.

### BRIEF DESCRIPTION OF THE DRAWINGS

FIG. 1 shows a comparison of an amino acid sequence of an enzyme according to the present invention and an amino acid sequence of a catalytic site of endoglucanase of acidothermus cellulolyticus.

FIG. 2 shows the relative activity of the enzyme of the present invention at various pH levels.

FIG. 3 shows the relative activity of the enzyme of the present invention incubated at various temperatures.

FIG. 4 shows the remaining activity after the enzyme of the present invention is heat-treated at 97° C.

FIG. 5 shows the relationship between the concentration of reducing sugar and the relative viscosity in the cases of the enzyme of the present invention and known endoglucanase.

### DESCRIPTION OF THE PREFERRED EMBODIMENTS

Embodiments of the present invention will be described in detail below.

The term “cellulose” used herein includes avicel, carboxymethyl cellulose, and lichenan.

The term “endoglucanase” means any endoglucanase, or an enzyme that catalyzes selective hydrolytic splitting of β-1,4-D glucosidic bonds between D-glucoses of cellulose. The enzyme commission (EC) number of such endoglucanase is 3.2.1.4.

The thermophilic endoglucanase of the present invention is stable at around 90° C. to 100° C. which is an optimum growth temperature range. Preferably, the endoglucanase of the present invention has the following properties:

(a) a molecular weight of about 43 kilo daltons;

(b) an optimum temperature of 95° C. or more to 100° C. or less;

(c) an optimum pH of 5.4 to 6.0; and

(d) stability at about 97° C. for 3 hours.

The thermophilic endoglucanase of the present invention can be obtained from prokaryote, preferably from archaeon Pyrococcus, and more preferably from hyperthermophilic and sulfur metabolic Pyrococcus horikoshii (JCM Accession number 9974, JCM catalogue of strains, seventh edition, January, 1999) having an optimum growth temperature of 98° C.

The thermophilic endoglucanase of the present invention has a naturally occurring type of amino acid sequence as shown in SEQ ID NO: 2, for example. Alternatively, the thermophilic endoglucanase of the present invention includes polypeptide that comprises an amino acid sequence as shown in SEQ ID NO: 2 in which 1 to 100 amino acid residue(s) is/are missing, substituted, or added and that has a substantially similar activity to the naturally occurring type of thermophilic endoglucanase.

The thermophilic endoglucanase of the present invention can be a polypeptide having a functionally similar activity to the naturally occurring type polypeptide. This polypeptide has some variations. Such polypeptide can be obtained by alternating DNA encoding the desired polypeptide by a known genetic engineering technique and then expressing the altered DNA. Whether or not the polypeptide has a similar activity to a naturally occurring type polypeptide can be easily checked by assaying the activity.

The thermophilic endoglucanase of the present invention can be prepared as a fused protein. The fused protein contains a first peptide and a second peptide that are joined by a peptide bond. The first peptide is the thermophilic endoglucanase of the present invention, and the second peptide can be a certain protein or a fragment thereof. Examples of the certain protein that can be the second peptide include: beta galactosidase, glutathion S-transferase, luciferase, and horseradish peroxidase.

A nucleotide sequence encoding the thermophilic endoglucanase of the present invention can include a nucleotide sequence encoding an amino acid sequence as shown in SEQ ID NO: 1. Alternatively, it can include a nucleotide sequence encoding the amino acid sequence as shown in SEQ ID NO: 1 in which one or more amino acid residue(s) is/are missing, substituted, or added. Preferably, this nucleotide sequence is shown in SEQ ID NO: 1 from nucleotide 1 to nucleotide 1374 or in SEQ ID NO: 1 from nucleotide 85 to nucleotide 1374. Alternatively, the nucleotide sequence can be a polynucleotide sequence shown in SEQ ID NO: 1 from nucleotide 1 to 1377 or a nucleotide sequence encoding polypeptide that can be hybridized under a stringent condition with a sequence shown in SEQ ID NO: 1 from nucleotide 85 to nucleotide 1377 and that has a thermophilic endoglucanase activity.

In general, a method of artificially producing a variant of polynucleotide sequence and a homolog are known to those skilled in the art. Using such known techniques, a variant or a homolog having an activity shown by a naturally occurring type thermophilic endoglucanase can be produced, for example. Such variant and homolog are also included in the thermophilic endoglucanase of the present invention.

The phrase “to be hybridized under stringent conditions” used herein means to be hybridized at 65° C. in 0.2×SSC, for example.

The naturally occurring type thermophilic endoglucanase polynucleotide is obtained by: after incubating Pyrococcus horikoshii, searching gene sequences of the thermophilic bacteria of the present invention for genes that seem to be similar to an endoglucanase sequence of pyrococcus furiosus and to have an enzymatic activity of the present invention by a BLAST method; amplifying the genes in a PCR reaction; and then extracting a target gene (for example, a gene shown in SEQ ID NO: 1). After that, a transformant including a recombinant vector containing polynucleotide of the present invention is incubated and then a thermophilic endoglucanase is obtained from the culture medium.

The vector to be used is not particularly limited. For example, a usable vector is the one that can be replicated independently in a host cell or the one whose copy or copies can be inserted into a chromosome of the host cell. Any vector can be used, as far as it has an insertion site for the aforementioned DNA or a thermophilic endoglucanase gene and has an ability to express the DNA in the host cell.

The thermophilic endoglucanase gene to be inserted into the vector may be not only a cDNA but also a DNA that is synthesized as designed to encode an amino acid sequence predicted from the cDNA. A gene based on such amino acid sequence can be easily synthesized by annealing oligonucleotide synthesized by an automatic DNA synthesizer and linking it.

A powerful promoter that is usually used for expressing foreign protein can be used as a promoter for expressing the thermophilic endoglucanase gene. Also, a terminator can be inserted downstream of the thermophilic endoglucanase gene. Examples of terminators include trp, tac, lac, trc, λPL, T7 promoters and tpA, lpp, and T4 terminators.

In order to improve a translation efficiency, types and the number of SD sequence, a base composition of the area between the SD sequence and an initiation codon, and a sequence and length thereof can be suited for expressing a thermophilic endoglucanase gene.

The area between a promoter necessary for expressing the thermophilic endoglucanase and a translation initiation site can be prepared by the known PCR or chemical synthesizing technique.

The recombinant DNA of the present invention can be obtained by inserting a DNA including a gene encoding the thermophilic endoglucanase into a known expression vector according to a desired expression system by a known method. The expression vector used herein is desirably a multicopy vector.

Examples of the known vector to be used for preparing the recombinant DNA of the present invention include pUC18, pHSG299, and pET-11a.

Next, various transformants obtained by inserting the recombinant DNA or the expression vector thereinto will be described.

A conventionally known method can be used for introducing a recombinant vector obtained by inserting the recombinant DNA into an expression vector into a host cell. Examples of such method include: a competent cell method, protoplast method, calcium phosphate coprecipitation method, electroporation method, microinjection method, and liposome fusion method.

Examples of a host cell to be used as the transformant include E. coli JM109 strains such as recA, endA1, gyrA96, thi, hsdR17, supE44, relA1, and Δ(lac-proAB)/F′[traD36, proAB+, lacIq, lacZΔM15].

Other examples of a host cell to be used as the transformant include: Bacillus subtilis, yiest, and Aspergillus oryzae. The thermophilic endoglucanase of the present invention can be produced in a culture medium by using a protein secreting ability of these host cells. Also, host cell may include animal cell, plant cell, and insect cell.

A protein prepared in or secreted from such transformant is isolated and purified by a known method to obtain a target enzyme.

When E. coli is a host cell, an inactive aggregate or a protein inclusion body can be produced as a thermophilic endoglucanase gene product, and then activated by a certain method. After activation, the activated protein may be isolated and purified by a known method to thus obtain a target enzyme.

The transformant is grown in known culture medium. For example, E. coli is grown in nutrient medium such as LB medium or minimal medium such as M9 medium to which a carbon source, nitrogen source, vitamin source, and/or the like is/are added. Depending on the host cell, the transformant is grown at 16° C. to 42° C., preferably at 25° C. to 37° C. for 5 to 168 hours, and more preferably for 8 to 72 hours. The transformant can be grown in either shake culture or static culture, with stirring or aeration if necessary.

The thermophilic endoglucanase can be isolated and purified from extracts from the transformant by the following methods: a known salting-out method; a precipitation method such as isoelectric precipitation method and solvent precipitation method; a dialysis method; a filtration method using a molecular weight difference such as ultra filtration method or gel filtration method; a method using a specific affinity such as ion exchange chromatography; a method using a hydrophobic difference such as hydrophobic chromatography and reverse phase chromatography; affinity chromatography; SDS polyacrylamide electrophoresis; isoelectric focusing electrophoresis; and a combination thereof.

Alternatively, the obtained enzyme can be isolated and purified easily by heat treatment. A method of producing a thermophilic endoglucanase of the present invention by gene recombination will be described in detail in the following Examples. Many changes, modifications, variations, and other uses and applications of the subject invention will, however, become apparent to those skilled in the art after considering this specification and the accompanying drawings which disclose the preferred embodiments thereof. All such changes, modifications, variations, and other uses and applications which do not depart from the spirit and scope of the invention are deemed to be covered by the invention, which is to be limited only by the claims which follow.

This application claims priority from Japanese Patent Application No. 2001-349328 and No. 2002-295578, which are incorporated herein by reference.

### EXAMPLES

The invention will be more clearly understood by referring to the examples which follow. These examples, which illustrate specific embodiments of the present invention, should not be construed to limit the invention in any way.

### Example 1

Incubation of Pyrococcus horikoshii

The pyrococcus horikoshii is a known thermophilic archaeon, and the incubation conditions thereof are also known. In this specification, one of the pyrococcus horikoshii is taken as an example.

13.5 g of NaCl, 4 g of Na₂SO₄, 0.7 g of KCl, 0.2 g of NaHCO₃, 0.1 g of KBr, 30 mg of H₃BO₃, 10 g of MgCl₂.6H₂O, 1.5 g of CaCl₂, 25 mg of SrCl₂, 1.0 ml of resazurin solution (0.2 g/L), 1.0 g of yeast extract, and 5 g of bactopepton were dissolved in 1 litter of distilled water to adjust the mixture to pH 6.8, and then the solution was sterilized by pressure. Dry-sterilized element sulfur was added to the mixture to prepare medium culture with 0.2% sulfur. The medium culture was saturated with argon gas so as to make it anaerobic, and then Pyrococcus horikoshii 0T 3 was implanted therein. When a Na₂S solution was added to the medium, it was not turned into pink. Thus, it was confirmed that the medium culture became anaerobic. The culture solution was incubated at 95° C. for 2 to 4 days.

### Example 2

Preparation of DNA Chromosome

After incubation, the culture solution was centrifuged at 5000 rpm for 10 minutes to collect bacteria. The bacteria was washed twice with a 10 mM Tris(pH 7.5)-1 mM EDTA solution, and then sealed in an InCert Agarose (FMC) block. The block was treated in a 1%N-lauroyl sarcosine-1 mg/ml protease K solution to seal DNA chromosome into the Agarose block.

### Example 3

Preparation of Library Clone Containing DNA Chromosome

The DNA chromosome obtained in Example 2 was cut into fragments using a restriction enzyme HindIII and fractionated by agarose gel electrophoresis. The DNA fragments of about 40 kb length were extracted from the gel. These DNA fragments were ligated using T4 ligase to Bac vector pBAC108L or pFOS1 that was completely cut by the restriction enzyme HindIII. When the former vector pBAC108L was used, the DNA was introduced into E. coli by electroporation immediately after ligation. Alternatively, when the latter vector pFOS1 was used, the ligated DNA was packaged into λ-phage particles using GIGA Pack Gold (Stratagene) in a test tube, and then the λ-phage was infected to E. coli to introduce the DNA into the E. coli. Chloramphenicol-resistant colonies of E. coli obtained by these methods were used as BAC library or Fosmid library. Suitable clones for covering JCM9974 chromosome were screened from the library and then ordered.

### Example 4

Determination of Base Sequence of BAC Clone or Fosmid Clone

A base sequence of the sequenced BAC clone or Fosmid clone was determined by the following method. The BAC clone DNA or Fosmid clone DNA collected from E. coli was subjected to ultrasonication to cut it into DNA fragments, and then fractionated by agarose gel electrophoresis. The DNA fragments of 1 kb or 2 kb length were extracted from the gel. The fragments were inserted into the restriction enzyme site of HincII of a plasmid vector pUC118 by shotgun, and thus 500 shotgun clones were prepared from BAC clone or Fosmid clone. The base sequence of each shotgun clone was determined using the ABI Prism 373 or 377 sequencer Perkinelmer. Then all the base sequences of the BAC clone or Fosmid clone were determined by compiling the base sequences of the shotgun clones using the automated sequencing program Sequencher.

### Example 5

Identification of Genes Encoding Endoglucanase

The base sequences of the BAC clone or Foemid clone were analyzed using a large scale computer to identify a gene encoding endoglucanase (SEQ ID NO: 1). This gene was composed of 1377 base pairs in a range from an initiation codon to a termination codon, and thus the expected number of residues of amino acid sequences of the endoglucanase was 458 residues.

### Example 6

Construction of Expression Plasmid

In order to construct restriction enzyme sites (NdeI and XhoI) at the ends of a structural gene region, the following two kinds of DNA primers were synthesized on the basis of genome analyzing data available from Independent Administration Institute (a government agency) System, National Institute of Technology and Evaluation (NITE): primer 1:5′-TTTTGAATTCTTTCATATGGAGGGGAATACTATTCTTAAAATC-3′ (upper primer, SEQ ID NO: 3); and primer 2:5′-TTTTTCTAGATTTGGATCCTTTGGGCTACCTGGGAGCCCTTCTTAA-3′ (lower primer, SEQ ID NO: 4). Using this primer pair, the restriction enzyme sites were added to the ends of the gene by the PCR (polymerase chain reaction) technique. After that, the structural gene was completely cut by the restriction enzymes (NdeI and XhoI) at 37° C. for 2 hours and then purified.

After the pET-11a plasmid (Novagen) was cut by the restriction enzymes NdeI and XhoI and purified, it was reacted with the aforementioned structural gene and T4 ligase at 16° C. for 2 hours for ligation. The ligated DNA was introduced into a competent cell of E. coli-XL2-BlueMRF′ (Stratagene) to obtain transformant colonies. The tranformant colonies were purified by alkali treatment, and thus expression plasmid was obtained.

### Example 7

Expression of Recombination Gene

Competent cells of E. coli BL21(DE3)pLysS (Novagen) were lysed and then 0.1 mL of them was added to a Falcon tube. After adding 0.005 mL of the expression plasmid solution in the tube, the tube was left in ice bath for 30 minutes and heat-shocked at 42° C. for 30 seconds. Then, 0.9 mL of SOC medium was added to the tube and incubated on shake at 37° C. for an hour. After that, the liquid culture was spread on a 2YT agar plate containing ampicillin and incubated overnight at 37° C. Thus, a transformant was obtained.

After the transformant was added to 1L of the 2YT agar plate containing ampicillin and cultured until the absorbance reached 1 at 600 nm, 10 mL of IPTG (Isopropyl-b-D-thiogaractopyranoside) 100 mM solution was added to the culture medium and incubated for 6 hours. Then the culture medium was centrifuged at 6,000 rpm for 20 minutes to collect bacteria.

### Example 8

Purification of Thermophilic Enzyme

Twice as much alumina as the collected bacteria was added to the bacteria so as to grind the bacteria. Then, five times as much 10 mM tris hydrochloride buffer (pH 8.0) was added to prepare a suspension. The suspension was heated at 85° C. for 30 minutes and then centrifuged at 11,000 rpm for 20 minutes. Supernatant was loaded onto a HiTrapQ column (Pharmacia), and thus active fractions were obtained.

### Example 9

Various Properties of Enzyme

(1) Molecular Weight and Amino Acid Sequence

SDS-PAGE was carried out using a Phast system (Pharmacia) on a gel gradient of 10 to 15%. The active fractions showed a single band with a suitable molecular weight (43 kD) calculated from the amino acid sequence, so that they were used for analyzing properties of an enzyme.

In addition to the SDS-PAGE, high performance liquid chromatography (HPLC) was carried out using a TSK gel G3000 SW_{XL} column (TOSOH, Tokyo) to determine a molecular weight. Elution was carried out with 50 mM sodium phosphate buffer (pH 6.8) containing 0.3 M NaCl at a flow rate of 0.8 ml/min at room temperature. The eluted protein was detected by UV absorbance of 280 nm. It was confirmed by both the SDS-PAGE and the gel filtration that the enzyme purified by the HiTrapQ column had a molecular weight of 43 kD.

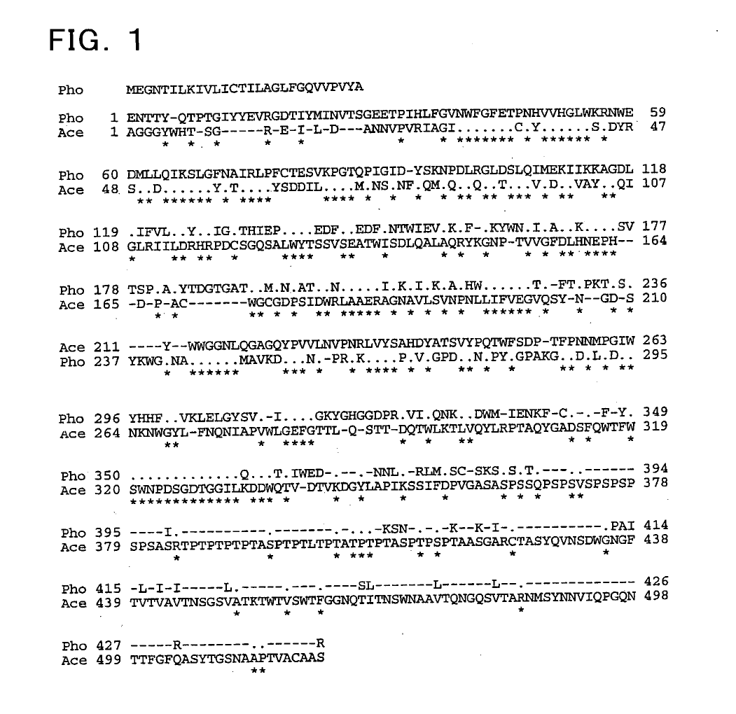

Since this value was smaller than the value (about 52 kD) calculated on the basis of 458 residues that was estimated from the open reading frame of the gene sequence, it was suspected that N-terminal and/or C-terminal residues were missing in the protein. In order to make this clear, an amino acid analysis of the N-terminal region was performed on the purified protein. The result indicated that the first 28 amino acid residues that was listed as a SEQ ID NO. 2 were missing. Accordingly, the sequence corresponding to the first 28 amino acid residues had characteristics that was unique to signal peptide sequences. Judging from the measured molecular weight, it was suspected that some residues of the C-terminal region were also missing. The sequence corresponding to a catalystic site from the 29th residue to C-terminal residue had a homology of 43% to a catalystic site of endoglucanase of Acidothermus cellulolyticus (FIG. 1). In FIG. 1, each upper sequence shows the amino acid sequence of endoglucanase (EGPh) of the present invention and each lower sequence shows a catalystic site of endoglucanase (EGAc) of Acidothermus cellulolyticus.

(2) Optimum pH

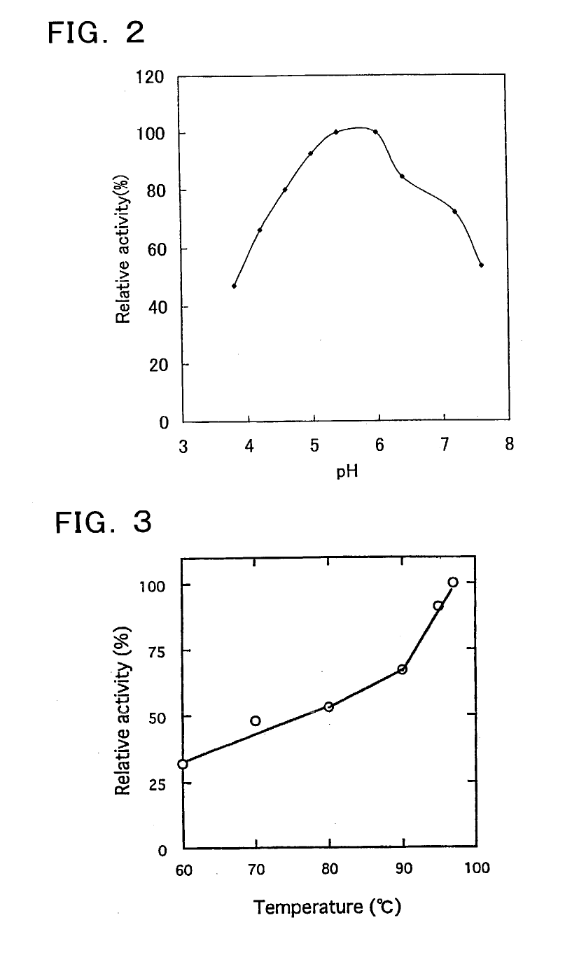

An optimum pH of the enzyme activity was assayed by measuring the initial rate of hydrolytic activity of the enzyme at 85° C. in a 0.5% carboxymethyl cellulose solution that was adjusted to pH 4 to pH 9 with 100 mM sodium acetate buffer, 100 mM phosphate buffer, and 100 mM borate buffer. The activity was assayed by determining the quantity of reducing terminal resulting from hydrolysis using the Somogyi-Nelson method. As shown in FIG. 2, the activity reached its maximum rate at around pH 5.6, and the rates close to the maximum rate were obtained at pH 5.4 to pH 6.0. Therefore, it was concluded that the optimum pH was pH 5.4 to pH 6.0.

(3) Optimum Temperature

In the same manner as the item (2), the relative activity to various temperatures were measured according to the Somogyi-Nelson method by reacting a certain amount of the enzyme with 0.5% carboxymethyl cellulose in 100 mM sodium acetate buffer (pH 5.6) for 15 minutes. Since the relative activity reached its maximum at around 97° C. as shown in FIG. 3, it was concluded that a maximum activity (optimum temperature) was 97° C. or more.

(4) Heat Resistance (Thermostability)

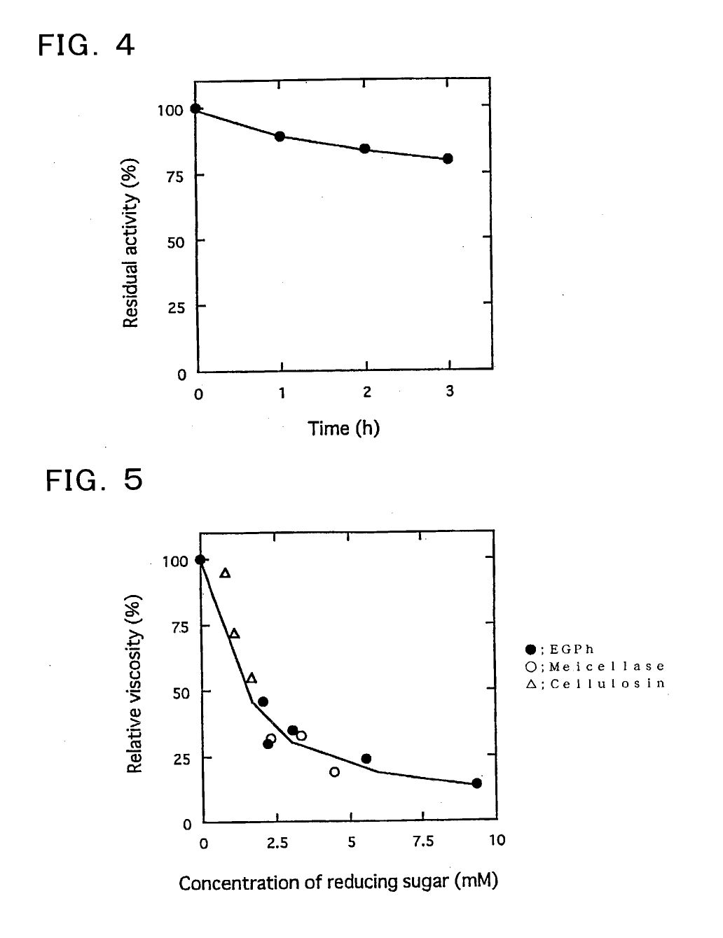

Residual activity was measured by incubating aliquats of the enzyme solution (0.1 mg/mL) at 97° C. in 100 mM sodium acetate buffer (pH 5.6) for 3 hours and then lowering the temperature to 85° C. As shown in FIG. 4, residual activity was 80%.

(5) Determination of the Mode of Hydrolysis of the Enzyme

In order to determine the mode of hydrolysis of EGPh of the present invention, the viscosity of a hydrolysis product by the EGPh was measured and compared with viscosities of the hydrolysis products by endo-type enzymes such as Cellulosin AC-8 (Hankyu Kyoei Bussan Co., Ltd.) and Meicelase SP-100 (Meiji Seika Kaisha, Ltd.) which are known as endo-type glucanase. 0.5% carboxymethyl cellulose solutions were reacted in 100 mM sodium acetate buffer (pH 5.6) with a certain amount of the EGPh at 90° C. for 15 minutes and with Cellulosin and with Meicelase at 40° C. for 15 minutes, respectively. Each enzyme was added at a concentration of 20 μg/ml.

In the case of endo-type enzyme, since the hydrolysis of cellulose begins from the inside, cellulose having a large molecular weight (namely, a viscosity) is sharply decreased at first but gradually decreased as time goes by. Therefore, the hydrolysis product, or glucose, is gradually increased at first but is sharply increased as time goes by. On the contrary, in the case of exo-type enzyme, since the hydrolysis of cellulose begins at the end thereof, a decrease of cellulose having a large molecular weight (namely, a decrease of viscosity) is moderate and constant.

As shown in FIG. 5, the viscosity of cellulose in the case of the EGPh was sharply decreased at first and then moderately decreased as in the case of Cellulosin AC-8 and Meicelase SP-100. Thus, it was concluded that the endoglucanase (EGPh) of the present invention was an endo-type cellulose.

(6) Substrate Specificity

The substrate specificity was measured using carboxymethyl cellulose (CMC), Avicel SF (Asahi Kasei), lichenan (Nacalai Tesque), and cell-oligomer (cellobiose to cellopentaose; Seikagaku Corporation) as a substrate. Further, curdlan, xylan, and xyloglucan (Nacalai Tesque, Inc.) were also used as a substrate. An enzyme solution (0.01 μg/mL) was incubated in 100 mM sodium acetate buffer (pH 5.6) containing various substrates (2 mM of cello-oligomer and 0.5% of other substrates) at 85° C. for an hour, and then catalystic activity was assayed.

The activity was assayed by measuring the amount of reducing sugar resulting from hydrolysis by the Somogyi-Nelson method. As a result, carboxymethyl cellulose (CMC), Avicel SF, lichenan, and cell-oligomer were hydrolyzed, but no activity was detected toward curdlan, xylan, and xyloglucan. Results of assays are shown in Table 1.

TABLE 1
Substrate specificity of EGPh

| Substrate | | Specific activity (kcat) (per sec) |
|---|---|---|
| CMC | (0.25% w/v) | 1.286 ± 0.082 |
| Avicel SF | (0.25% w/v) | 0.212 ± 0.050 |
| Lichenan | (0.25% w/v) | 0.565 ± 0.147 |
| Xylan | (0.25% w/v) | ND |
| Xyloglucan | (0.25% w/v) | ND |
| Curdlan | (0.25% w/v) | ND |
| Cellobiose | (2 mM) | 0.0047 ± 0.0003 |
| Cellotriose | (2 mM) | 0.0076 ± 0.0019 |
| Cellotetraose | (2 mM) | 0.0250 ± 0.0001 |
| Cellopentaose | (2 mM) | 0.0252 ± 0.0017 |

What is claimed is:

1. A thermophilic endoglucanase having:
(a) a molecular weight of about 43 kilo daltons;
(b) an optimum temperature of 95° C. or more:
(c) an optimum pH of 5.4 to 6.0; and
(d) stability at about 97° C. for 3 hours.

2. The thermophilic endoglucanase according to claim 1, derived from prokaryote.

3. The thermophilic endoglucanase according to claim 1, derived from Pyrococcus.

4. The thermophilic endoglucanase according to claim 1, derived from Pyrococcus horikoshii.

5. A thermophilic endoglucanase comprising an amino acid sequence as shown in positions 1-458 of SEQ ID NO: 2.

6. A thermophilic endoglucanase having a thermophilic endoglucanase activity and comprising an amino acid sequence as shown in positions 1-458 of SEQ ID NO: 2 in which 1 to 100 amino acid residue(s) is/are missing, substituted, or added.

7. A thermophilic endoglucanase comprising an amino acid sequence as shown in positions 29-458 of SEQ ID NO: 2.

8. A thermophilic endoglucanase having a thermophilic endoglucanase activity and comprising an amino acid sequence as shown in positions 29-458 of SEQ ID NO: 2 in which 1 to 100 amino acid residue(s) is/are missing, substituted, or added.

9. The thermophilic endoglucanase according to any one of claims 5 to 8, derived from prokaryote.

10. The thermophilic endoglucanase according to any one of claims 5 to 8, derived from Pyrococcus.

11. The thermophilic endoglucanase according to any one of claims 5 to 8, derived from Pyrococcus horikoshii.

12. A DNA comprising a polynucleotide sequence encoding the thermophilic endoglucanase according to claim 5.

13. A DNA comprising a polynucleotide sequence encoding the thermophilic endoglucanase according to claim 6.

14. A DNA comprising a polynucleotide sequence encoding the thermophilic endoglucanase according to claim 7.

15. A DNA comprising a polynucleotide sequence encoding the thermophilic endoglucanase according to claim 8.

16. A DNA comprising a polynucleotide sequence encoding the thermophilic endoglucanase according to claim 11.

17. A DNA comprising a polynucleotide sequence as shown in SEQ ID NO: 1 from nucleotide 1 to nucleotide 1374.

18. A DNA comprising a polynucleotide sequence as shown in SEQ ID NO: 1 from nucleotide 85 to nucleotide 1374.

19. A DNA for encoding a polypeptide, wherein said polypeptide can be hybridized with a polynucleotide sequence as shown in SEQ ID NO: 1 from nucleotide 85 to nucleotide 1377 under stringent conditions and has a thermophilic endoglucanase activity.

20. A recombinant vector comprising the DNA according to any one of claims 12 to 19.

21. A transformant transformed by the vector according to claim 20.

22. The transformant according to claim 21 whose host is a bacteria cell, animal cell, plant cell, or insect cell.

23. A method of producing a polypeptide having a thermophilic endoglucanase activity, comprising the steps of:
incubating the transformant according to claim 22; and
obtaining a thermophilic endoglucanase from a culture.

24. An expression vector comprising the DNA according to any one of claims 12 to 19 in a site to be controlled by a promoter for expressing a thermophilic endoglucanase and having an ability of expressing a polypeptide that has a thermophilic endoglucanase activity in a host cell.

25. A transformant transformed by the vector according to claim 24.

26. The transformant according to claim 25 whose host is a bacteria cell, animal cell, plant cell, or insect cell.

27. A method of producing a polypeptide having a thermophilic endoglucanase activity, comprising the steps of:
incubating the transformant according to claim 26; and
obtaining a thermophilic endoglucanase from a culture.
